(19) United States
(12) Patent Application Publication
(10) Pub. No.: US 2025/0345638 A1
(43) Pub. Date: Nov. 13, 2025

(54) SYSTEM FOR AND METHOD OF PLANNING AND REAL-TIME NAVIGATION FOR TRANSCRANIAL FOCUSED ULTRASOUND STIMULATION

(71) Applicant: The General Hospital Corporation, Boston, MA (US)

(72) Inventors: Bastien Guerin, Charlestown, MA (US); Aapo Nummenmaa, Charlestown, MA (US); Mohammad Daneshzand, Charlestown, MA (US)

(21) Appl. No.: 18/862,300

(22) PCT Filed: May 2, 2023
(86) PCT No.: PCT/US2023/066467

§ 371 Date: Nov. 1, 2024

Related U.S. Application Data
(60) Provisional application No. 63/337,134, filed on May 1, 2022.

(51) Int. Cl. A61N 7/02; A61N 7/00; G01R 33/56; G16H 20/30

(52) U.S. Cl. CPC A61N 7/02; G01R 33/5608; G16H 20/30; A61N 2007/0026; A61N 2007/025

(57) ABSTRACT

A system for planning and real-time navigation for transcranial focused ultrasound stimulation (tFUS) including receiving an image of a head of a subject, an acoustic beam profile simulation module configured to generate a subject-specific set of acoustic beam profiles based on the subject's head; subject-specific set of acoustic beam profiles configured to account for acoustic propagation effects through the subject's skull; a planning module coupled to the acoustic beam profile simulation module configured to generate an acoustic intensity scalp map for a target region and to generate a three-dimensional (3D) visualization of a selected beam profile from the subject-specific set of acoustic beam profiles; and a real-time navigation module coupled to the acoustic beam profile simulation module configured to generate a real-time 3D visualization of an acoustic beam for tFUS for a current position of a transducer around the head of the subject based on current position data and the subject-specific set of acoustic beam profiles.

### CROSS-REFERENCE TO RELATED APPLICATIONS

This application is based on, claims priority to, and incorporates herein by reference in its entirety U.S. Ser. No. 63/337,134 filed May 1, 2022, and entitled “System for and Method of Transcranial Focused Ultrasound Stimulation.”

### STATEMENT REGARDING FEDERALLY SPONSORED RESEARCH OR DEVELOPMENT

This invention was made with government support under 2019A014702 awarded by the National Institutes of Health. The government has certain rights in the invention.

### FIELD

The present disclosure relates generally to transcranial focused ultrasound stimulation (tFUS) and more particularly to subject-specific planning and real-time navigation of tFUS accounting for non-uniform propagation through structures such as, for example, the skull.

### BACKGROUND

Transcranial Focused Ultrasound Stimulation (tFUS) is an emerging non-invasive brain neurostimulation technology that allows targeting deep brain structures with high spatial precision. The 3D focusing capabilities of tFUS enables selective stimulation of deep targets associated with, for example (but not limited to), the treatment of major depressive disorder, obsessive compulsive disorder (OCD) and disorders of consciousness. Neuromodulation with tFUS is quickly gaining popularity, with many ongoing studies attempting to assess its clinical efficacy. However, distortion of the tFUS beam by structures (e.g., the skull, tissues) located between the transducer and the target is a significant barrier to accurate delivery of the acoustic energy at the correct location in the brain, and therefore a difficulty for translation of this novel neurotherapeutics modality in clinics. In other words, accurate targeting of a specific nucleus or other brain region with tFUS requires subject-specific modeling of the acoustic beam distortion by structures such as, for example, the skull.

### SUMMARY

In accordance with an embodiment, a system for planning and real-time navigation for transcranial focused ultrasound stimulation (tFUS) includes an input for receiving an image of a head of a subject and an acoustic beam profile simulation module coupled to the input and configured to generate a subject-specific set of acoustic beam profiles for a plurality of transducer locations based on the image of the head of the subject. The subject-specific set of acoustic beam profiles is configured to account for acoustic propagation effects through the subject's skull. The system further includes a planning module coupled to the acoustic beam profile simulation module and configured to generate an acoustic intensity scalp map for a target region based on the subject-specific set of acoustic beam profiles and to generate a three-dimensional (3D) visualization of a selected beam profile from the subject-specific set of acoustic beam profiles, and a real-time navigation module coupled to the acoustic beam profile simulation module and configured to generate a real-time 3D visualization of an acoustic beam for tFUS for a current position of a transducer around the head of the subject based on current position data and the subject-specific set of acoustic beam profiles.

In accordance with another embodiment, a method for planning a transcranial focused ultrasound stimulation (tFUS) study for a subject includes retrieving a pre-calculated subject-specific set of acoustic beam profiles for a plurality of transducer locations. The subject-specific set of acoustic beam profiles is configured to account for acoustic propagation effects through the subject's skull. The method further includes generating an acoustic intensity scalp map for a target region based on the subject-specific set of acoustic beam profiles, generating a three-dimensional (3D) visualization for a selected beam profile from the subject-specific set of acoustic beam profiles, and displaying the acoustic intensity scalp map and the 3D visualization of the selected beam profile on a display.

In accordance with another embodiment, a method for real-time navigation for a transcranial focused ultrasound stimulation (tFUS) study of a subject includes retrieving a pre-calculated subject-specific set of acoustic beam profiles for a plurality of transducer locations. The subject-specific set of acoustic beam profiles is configured to account for acoustic propagation effects through the subject's skull. The method further includes receiving current position data for a transducer indicating a current position of the transducer around the head of the subject, generating a real-time 3D visualization of an acoustic beam for tFUS for the current position of a transducer around the head of the subject based on the current position data and the subject-specific set of acoustic beam profiles, and displaying the real-time 3D visualization of an acoustic beam for tFUS for the current position of a transducer around the head of the subject.

### BRIEF DESCRIPTION OF THE DRAWINGS

The present disclosure will hereafter be described with reference to the accompanying drawings, wherein like reference numerals denote like elements.

FIG. 1 is a block diagram of a system for planning and real-time navigation for transcranial focused ultrasound stimulation (tFUS) in accordance with an embodiment;

FIG. 2 is a block diagram of an acoustic beam profile simulation module in accordance with an embodiment;

FIG. 3 illustrates an example graphical user interface for an acoustic beam profile simulation module in accordance with an embodiment;

FIG. 4 illustrates an example graphical user interface for a planning module in accordance with an embodiment;

FIG. 5 illustrates an example graphical user interface for a real-time navigation module in accordance with an embodiment;

FIG. 6 illustrates an example method for determining acoustic beam profiles in accordance with an embodiment;

FIG. 7 illustrates method for planning for a transcranial focused ultrasound stimulation (tFUS) study of a subject in accordance with an embodiment;

FIG. 8 illustrates a method for real-time navigation for a transcranial focused ultrasound stimulation (tFUS) study of a subject in accordance with an embodiment;

FIG. 9 is a block diagram of an example computer system in accordance with an embodiment; and

FIG. 10 is block diagram of an example focused ultrasound system in accordance with an embodiment.

### DETAILED DESCRIPTION

The present disclosure describes systems and methods for subject-specific planning and real-time navigation for transcranial focused ultrasound stimulation (tFUS) using pre-calculated set of acoustic beam profiles that account for non-uniform propagation through the skull. The disclosed systems and methods can provide a tool for accurate calculation of the tFUS acoustic beam distortions by the skull based on an MRI image of the subject, for example, an MRI image of the head of the subject. In some embodiments, a system for planning and real-time navigation of tFUS can include an acoustic beam profile simulation module or tool, a planning module or tool and a real-time navigation module or tool. The acoustic beam profile simulation module may be configured to perform a pre-calculation of a subject specific basis-set of acoustic beams (e.g., acoustic beam profiles) for a plurality of locations around the subject's scalp and the subject specific basis-set of acoustic beams can advantageously account for complex acoustic propagation effects through a structure, for example, the skull of the subject. In some embodiments, the subject specific basis-set of acoustic beams can be pre-calculated for a plurality of transducer locations around the subject's scalp. In some embodiments, the subject specific basis-set of acoustic beams may be pre-calculated for a plurality of ultrasound excitations or basis functions around the subject's scalp. The ultrasound excitation or basis functions can include, for example, point sources, random sources, and plane waves. The subject-specific basis set of acoustic beam profiles can provide a discretized solution set, for example, for precise targeting and dosing of tFUS studies. In some embodiments, the pre-calculated subject specific basis set of acoustic beams for a plurality of locations can be utilized by the planning module to, for example, generate an acoustic intensity scalp map for a target region (e.g., a target region in the brain of the subject such as the thalamus or the amygdala) for all of the transducer or point source locations. In some embodiments, the pre-calculated subject specific basis set of acoustic beams for a plurality of locations can be utilized by the real-time navigation module to generate real-time three-dimensional (3D) visualizations of tFUS acoustic beams for a current position of a physical transducer (e.g., as the physical transducer is moved around the subject's head) so that the real-time 3D visualization of the acoustic beam accounts for the beams deformations (i.e., non-uniform propagation) of, for example, the subject's skull. In some embodiments, the disclosed systems and methods for subject-specific planning and real-time navigation for transcranial focused ultrasound stimulation (tFUS) may be used in conjunction with other stimulation modalities such as Transcranial Magnetic Stimulation (TMS).

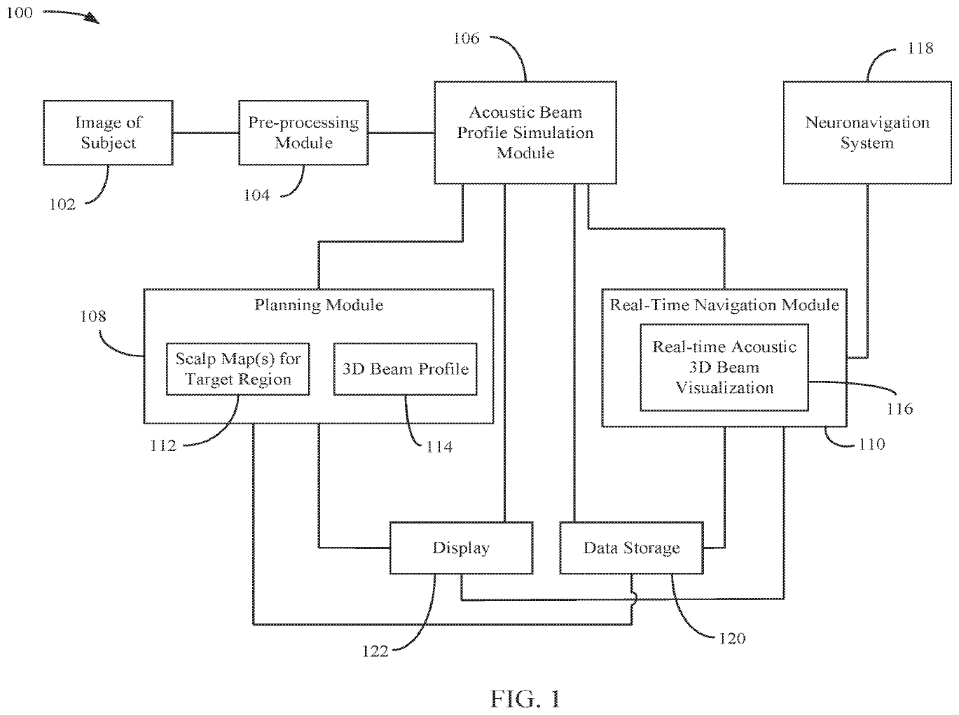

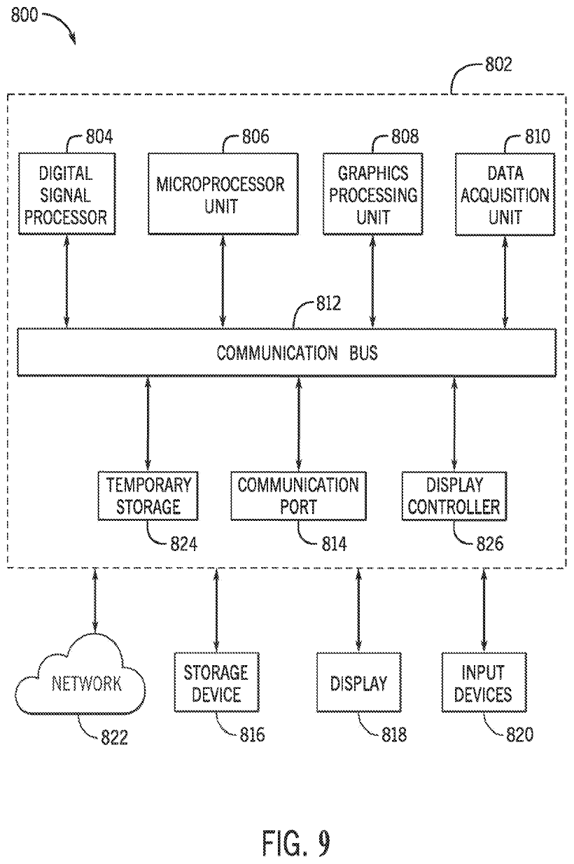

FIG. 1 is a block diagram of a system for planning and real-time navigation for transcranial focused ultrasound stimulation (tFUS) in accordance with an embodiment. System 100 can include an input image 102 of a subject, a pre-processing module 104, an acoustic beam profile simulation module 106, a planning module 108, a real-time navigation module 110, data storage (or memory) 120 and a display 122. The input image 102 of the subject can be a magnetic resonance (MR) image acquired using, for example, an MRI system using various MR imaging (MRI) acquisition techniques. In some embodiments, the input image 102 of the subject may be a CT image acquired using, for example, a CT system using various CT acquisition techniques. In some embodiments, the input image 102 of the subject may be retrieved from data storage (or memory) of an imaging system (e.g., an MRI system or a CT system) or data storage of other computer systems (e.g., storage device 816 of computer system 800 shown in FIG. 9). In some embodiments, the subject's MR and CT images (e.g., either estimated from the MRI image or measured/acquired directly) may be displayed on a display 122 of the system 100 shown in FIG. 1 or a display of other computer systems (e.g., display 818 of the computer system 800 shown in FIG. 9).

In some embodiments, the input image 102 of the subject may be provided as input to the pre-processing module 104. In some embodiments, where the input image 102 of the subject is a MR image, the pre-processing module 104 may be configured to convert or transform the MR image into CT units (Hounsfield units (HUs) using a pseudo-CT technique to generate a pseudo-CT image that accounts for the subject's individual skull geometry. In some embodiments, where a pseudo-CT image is generated, the pre-processing module 104 may also be configured to determine or estimate acoustic properties of the subject's skull based on the pseudo-CT image. For example, the acoustic properties of the skull may be estimated from the pseudo-CT image by using a scaling method that scales the Hounsfield units to acoustic properties (or parameters) or by using deep learning (e.g., a neural network). In some embodiments, an input MR image of the subject may not be pre-processed by the pre-processing module 104, but rather directly input to the acoustic beam profile simulation module 106. In some embodiments, the input image 102 of the subject (e.g., an MR image), the pseudo-CT image, and the estimated acoustic properties may be stored in data storage (or memory) 120 of system 100 or data storage of other computer systems (e.g., storage device 816 of computer system 800 shown in FIG. 9).

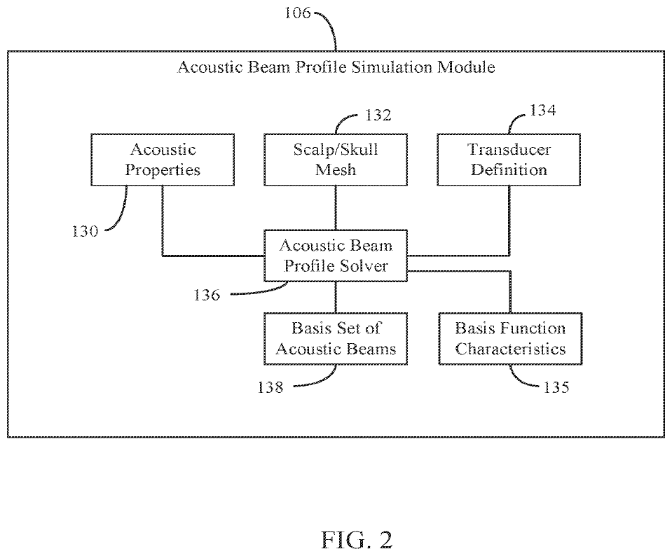

In some embodiments, the acoustic beam profile simulation module 106 may be configured to generate a subject-specific basis set of acoustic beam profiles. In some embodiments, the subject-specific basis set of acoustic beam profiles are calculated for a plurality of transducer locations around the scalp of the subject. In some embodiments, the subject-specific basis set of acoustic beam profiles can be decomposed on a basis set of pre-calculated ultrasound excitations such as, for example, point sources, plane waves, or other basis functions. The set of acoustic beam profiles can include calculated tFUS acoustic beams corresponding to placement of a transducer at hundreds of locations around the subject's scalp. The generated subject-specific basis set of acoustic beam profiles advantageously accounts for acoustic propagation effects through the subject's skull. In some embodiments, the acoustic beam profile simulation module can be configured to generate the subject-specific basis set of acoustic beam profiles using a pseudo-CT image and estimated acoustic properties. FIG. 2 is a block diagram of an acoustic beam profile simulation module in accordance with an embodiment. In FIG. 2, the acoustic beam profile simulation module 106 may be configured to create a mesh 132 representation of the scalp of the subject based on information from the MR image of the subject. For example, the scalp surface mesh 132 may be generated using a meshing routine. In some embodiments, the vertices of scalp/skull mesh 132 can represent the test transducer locations to be solved for by an acoustic beam profile solver 136. In some embodiments, around 1000 vertices can be used. More vertices may result in longer pre-computation times. In some embodiments, a smoothing technique (e.g., spatial smoothing may be used when generated the scalp surface mesh 132. In some embodiments, a user may select the number of vertices of the mesh 132 and may select a region outside of which vertices of the mesh are removed. For example, a user may manually select a 3D “box” outside of which mesh vertices may be rejected to avoid calculation of acoustic beams at locations that are not accessible to the operator or not relevant.

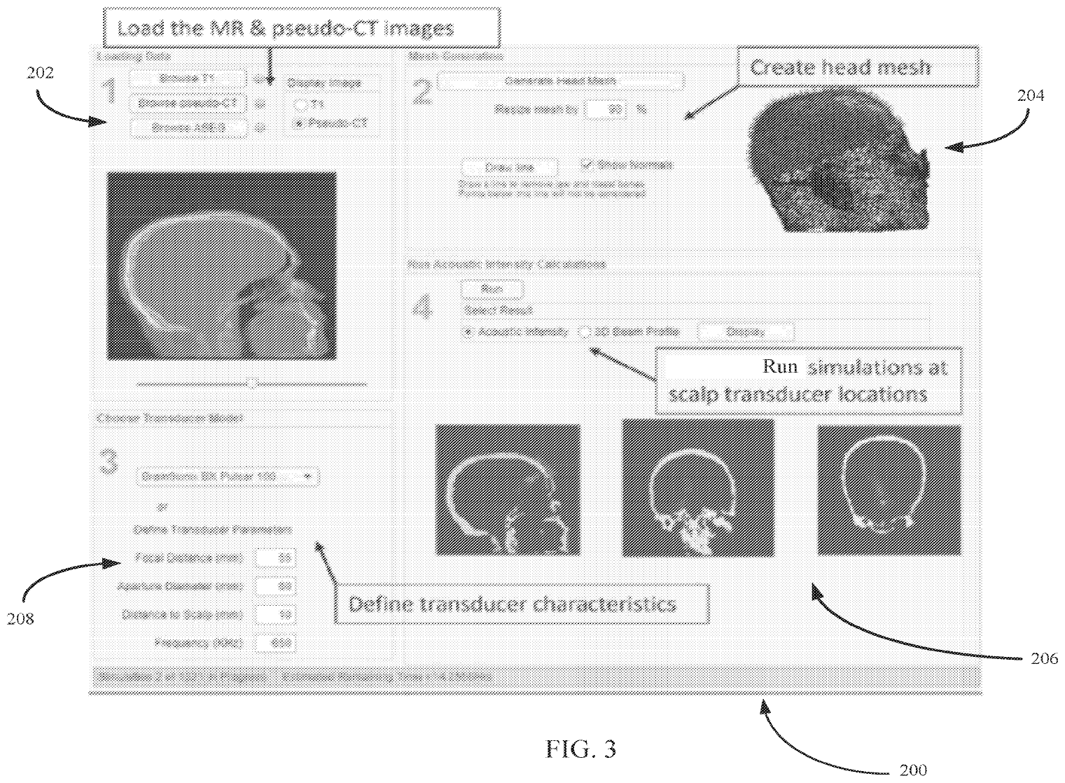

In some embodiments, for generating the set of acoustic beam profiles for a set of transducer locations, a transducer definition 134 may be provided or selected, for example, by a user or operator, that includes a plurality of transducer characteristics or parameters. In some embodiments, as described further below with respect to FIG. 3, the transducer parameters may be provided by selecting a transducer from a pre-populated list of transducers. In some embodiments, where the set of acoustic beam profiles are decomposed on a basis set of ultrasound excitations, a set of basis function characteristics 135 may be provided or selected, for example, by a user or operator. The acoustic properties 130, for example, determined by the pre-processing module 104, scalp/skull mesh 132, transducer definition 134, and set of basis functions characteristics 135 may be provided to the acoustic beam profile solver 136.

The acoustic beam profile solver 136 can be configured to compute acoustic beams created by the transducer, for example, at the vertices of the scalp mesh 132 or corresponding to a basis set of ultrasound excitations, to generate a subject-specific basis set of acoustic beam profiles 138. In some embodiments, the acoustic beam profile solver 136 is configured to compute transducer profiles at hundreds of locations around the subject's scalp. In some embodiments, the acoustic beam profile solver utilizes a fast method for computation of the tFUS beam profiles, for example, a hybrid angular spectrum (HAS) method, a finite element difference time domain accelerated on a graphical processing unit (GPU), a deep learning network, etc. Advantageously, the basis set of acoustic beams 138 accounts for non-linear acoustic propagation effects of structures, for example, the skull of the subject. In some embodiments, the acoustic beam profile solver 136 computes acoustic beams for a plurality of transducer locations around the subject's scalp based on, for example, the acoustic properties 130, the scalp mesh 132, and the transducer definition 134. . . . In some embodiments, the acoustic beam profile solver 136 computes acoustic beams for a basis set of ultrasound excitations (e.g., point sources, random sources, plane waves, etc.) at plurality (e.g., hundreds) of locations around the scalp based on, for example, the acoustic properties 130, the scalp mesh 132, and the basis function characteristics 135. An advantage of the approach utilizing the basis set of ultrasound excitations is that the excitations (e.g., point sources or other incident field shapes) form a convenient basis-set for the decomposition of arbitrary acoustic fields produced by sources placed outside the head. Accordingly, the acoustic field created by any arbitrary transducer geometry can be calculated very quickly by decomposition on the simulated ultrasound excitation basis set. As discussed further below with respect to the real-time visualization module 110, the rapidity of the decomposition (e.g., less than 1 second) can allow real time visualization of the tFUS beam as the user moves the physical transducer around the subject's head.

The subject-specific basis set of acoustic beam profiles 138 generated by the acoustic beam profile simulation module 106 may be stored in data storage (or memory) 120 of system 100 (shown in FIG. 1) or data storage of other computer systems (e.g., storage device 816 of computer system 800 shown in FIG. 9). As discussed further below, the basis set of acoustic beam profiles 138 can be provided to and subsequently used by the planning module 108 and the real-time navigation module 110. In some embodiments, the pre-computation performed by the acoustic beam profile simulation module 106 can be run before the tFUS study visit for the subject. If the pre-computation of the acoustic beam profile simulation module 106 is fast enough, i.e., a couple of minutes, the tFUS study visit can be the same day as the MRI scan visit used to acquire the MR image 102 of the subject used by the acoustic beam profile simulation module 106. In some embodiments, if the acoustic beam profile simulation module 106 requires many hours to run, the tFUS study visit may be planned another day. In some embodiments, the computation time of the acoustic beam simulation module 106 may be reduced (e.g., to a couple of minutes) using, for example, CPU acceleration and deep learning in order to allow same-day MRI and tFUS visits. As mentioned above, the input image 102 of the subject may be a CT image of the subject's head instead of an MRI. However, an MRI input image has an advantage of not increasing radiation exposure to the subject.

Returning to FIG. 1, in some embodiments, the acoustic intensity as well as the 3D beam profiles at every location generated by the acoustic beam profile simulation module 106 (e.g., as shown in FIG. 2) may be displayed as the simulation progresses. For example, the acoustic intensity as well as the 3D beam profiles at every location (e.g., a transducer location or a location of ultrasound excitation) may be displayed on a display 122 of the system 100 or a display of other computer systems (e.g., display 818 of the computer system 800 shown in FIG. 9).

In some embodiments, a system 100 may provide a graphical user interface to receive input from a user and to display various images and data to a user. FIG. 3 illustrates an example graphical user interface for an acoustic beam profile simulation module in accordance with an embodiment. The example graphical user interface (GUI) 200 is configured for the embodiment of the acoustic beam profile simulation module 106 described above with respect to FIG. 2A. In the GUI 200, a user may select an MR image of the subject and a corresponding pseudo CT image for input to the system 100. A scalp/skull mesh 204 generated by the acoustic beam profile simulation module 106 may be displayed. GUI 200 can also be configured to allow a user to select or enter transducer characteristic 208. The GUI 200 can also display the simulations 204 including, for example, the acoustic intensity and 3D beam profiles.

Returning to FIG. 1, the subject-specific basis set of acoustic beam profiles generated by the acoustic beam profile simulation module 106 may be provided to or retrieved by the planning module 108. In some embodiments the planning module 108 can be configured to use the subject-specific set of acoustic beam profiles to generate a scalp map 112 for a target region that can show the acoustic intensity in the brain target of interest for all of the transducer locations (or locations of ultrasound excitations around the scalp) simulated or modeled by the acoustic beam profile simulation module 106. The brain target or region of interest can depend on the specific tFUS study (or application) to be conducted for the subject, for example, some application may target the thalamus, other applications may target the amygdala, etc. In some embodiments, the target region can be selected or defined by a user. In some embodiments, a segmentation method may be used to segment regions that may be used as the target, for example, various cortical and subcortical regions. The planning module 108 can be configured to allow planning a tFUS session or study by mimicking the tFUS neuronavigation process before the subject actually comes to the study visit. In some embodiments, the planning module 108 can also be used after the tFUS study visit to better inform the result of that session.

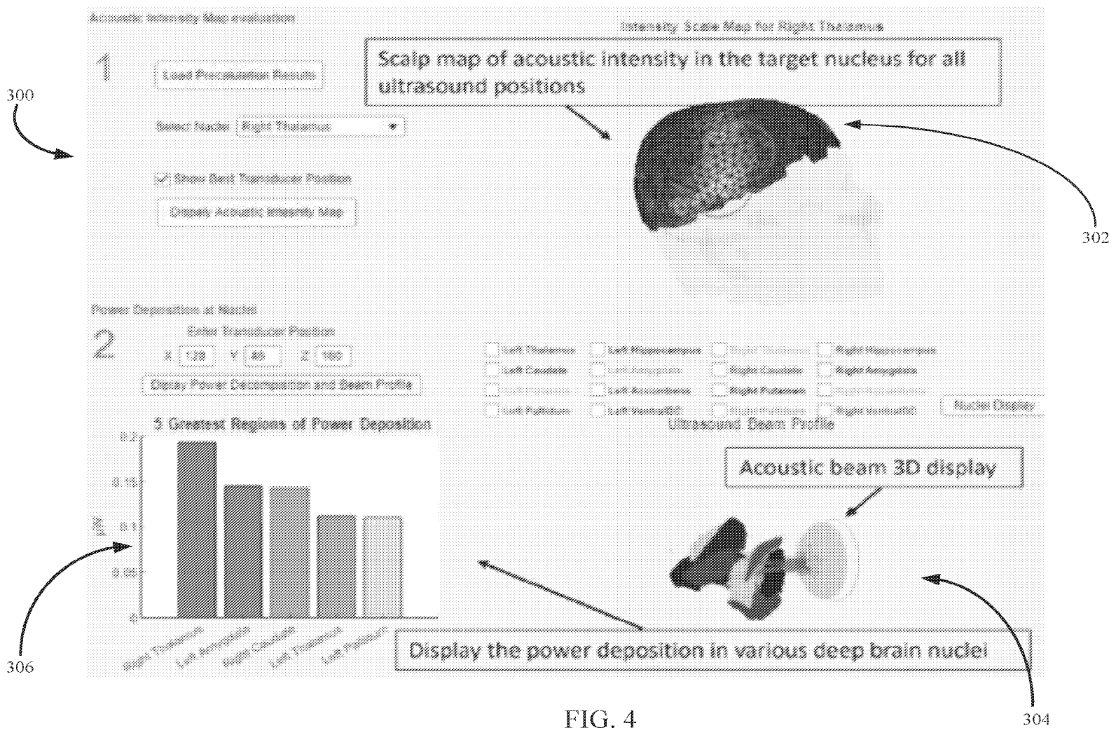

In some embodiments, the planning module 108 can allow a user to freely move a virtual transducer around a 3D scalp representation of the subject (e.g., a 3D mesh representation of the subject's scalp generated by the acoustic beam profile simulation module 106) and visualize the previously calculated 3D beam profiles 114 and acoustic focusing performance for specific transducer locations or point source locations. Accordingly, in some embodiments, the planning module 108 may be configured to generate a visualization of the acoustic 3D beam profile 114 (i.e., from the subject-specific basis set of acoustic beam profiles) for any selected transducer location. In some embodiments, the planning module 108 can also be configured to display a number of useful metrics, such as the average acoustic energy deposition in various nuclei as the user moves the virtual transducer at various test locations. FIG. 4 illustrates an example graphical user interface for a planning module in accordance with an embodiment. In the example graphical user interface (GUI) 300, a scalp map 302 of the acoustic intensity in a target nucleus for all transducer locations can be displayed. In addition, the GUI 300 includes a display of a 3D visualization of an expected acoustic beam (i.e., the pre-calculated acoustic beam from the subject-specific set of acoustic beam profiles) for a selected transducer (or point source) location. GUI 300 can also include a display (e.g., a graph) of metrics such as the power disposition 306 at nuclei.

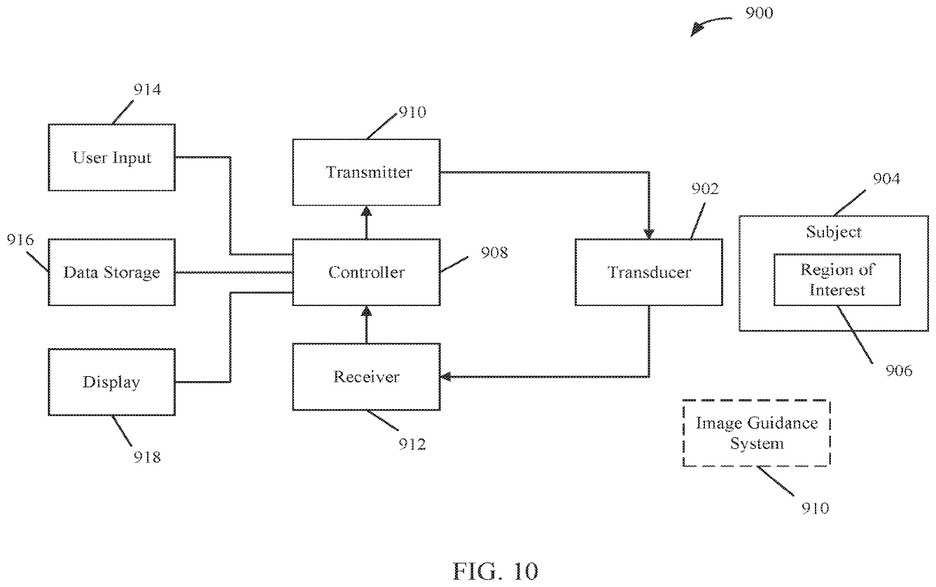

Returning to FIG. 1, the subject-specific basis set of acoustic beam profiles generated by the acoustic beam profile simulation module 106 may be provided to or retrieved by the real-time navigation module 110. In some embodiments, the real-time navigation module 110 can be configured to allow real-time visualization 116 of the tFUS beam as the physical transducer of a focused ultrasound system (e.g., focused ultrasound system 900 shown in FIG. 10) is freely moved around the subject's scalp. The visualization 116 of the tFUS beam may be generated using on the subject-specific set of acoustic beam profiles and based on current position data (e.g., potion and orientation) of the physical transducer. As mentioned above, in some embodiments, the acoustic beam profile simulation module 106 may simulate point sources or other basis set of ultrasound excitations (e.g., plane waves or random sources), rather than transducer locations, placed at hundreds of locations around the scalp. Advantageously, the acoustic field created by any arbitrary transducer geometry can be calculated very quickly by decomposition (e.g., less than 1 second) on a basis-set of ultrasound excitations which can help facilitate real-time visualization of the tFUS beam as the user moves the physical transducer around the subject's head. In some embodiments, a target region for visualization can be selected or defined by a user. In some embodiments, a segmentation method may be used to segment regions that may be used as the target, for example, various cortical and subcortical regions. The brain target or region of interest can depend on the specific tFUS study (or application) to be conducted for the subject, for example, some application may target the thalamus, other applications may target the amygdala, etc.

In some embodiments, the position data (e.g., position and orientation) for the physical transducer can be provided by a neuronavigation system 118 in communication with the real-time navigation module. In some embodiments, the real-time navigation module 110 may be implemented on the neuronavigation system 118. In some embodiments, the acoustic beam profile simulation module 106, the planning module 108 and the real-time navigation module 110 may be implemented on the neuronavigation system 118. The neuronavigation system 118 may be configured to track the movements of the physical transducer around the subject's scalp. The neuronavigation system 118 may be, for example, an optical neuronavigation tracking system. In some embodiments, the real-time navigation module 110 may be used, for example, right before a tFUS examination of the subject in order to position the transducer at an optimal position on the subject's scalp.

In some embodiments, the real-time navigation module 110 can be configured to register the subject's head to their anatomical MRI data (e.g., an input 102 MR image of the subject) using a tracker instrument placed on the head, which can be captured by, for example. a camera system of the neuronavigation system 118. A tracker can also be mounted on the tFUS transducer (e.g., transducer 1002 shown in FIG. 11) to measure the transducer's position with respect to the subject's head. The coordinates of the transducer may be communicated from the neuronavigation system 118 to the real-time navigation tool 110. The streamed tFUS tracker coordinates can be used by the real-time navigation module 110 to update and visualize 116 the acoustic beam of the tFUS, for example, the acoustic beam for a current position of the transducer. Accordingly, the real-time navigation tool 110 can be used to provide a real-time display of the tFUS beam as deformed by the skull, as the operator moves the transducer where the visualization of the tFUS acoustic beam is based on the position data from the neuronavigation system 118 and the set of acoustic beam profiles pre-computed by the acoustic beam profile simulation module 106. This rapid feedback to the user can allow testing many transducer positions and can possibly results in more optimal transducer positions which improved the accuracy of tFUS targeting compared to previous methods.

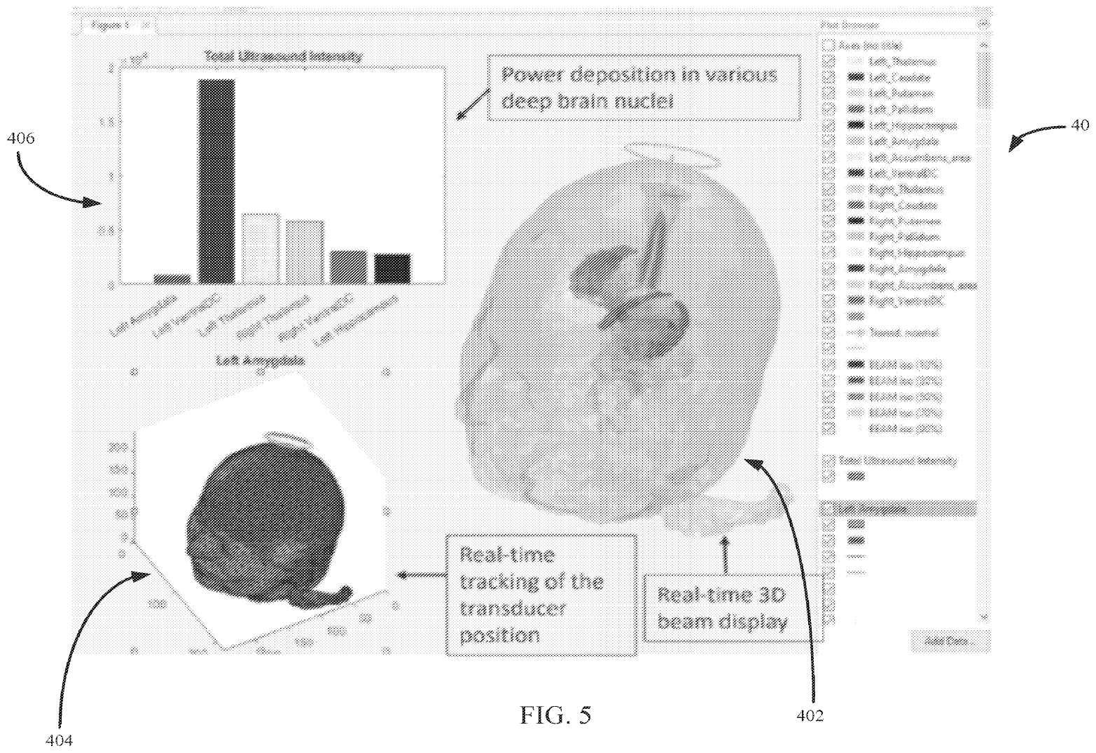

FIG. 5 illustrates an example graphical user interface for a real-time navigation module in accordance with an embodiment. In the example GUI 400, a real-time 3D display or visualization 402 of an acoustic beam is shown. The example GUI 400 may also be configured to display a real time tracking 404 of the transducer position based on the position data provided by the neuronavigation system 118. GUI 400 can also be configured to display data such as the power deposition in, for example, deep brain nuclei in the target region.

In some embodiments, the pre-processing module 104, the acoustic beam profile simulation module 106, the planning module 108, and the real-time navigation module 110 may be implemented on one or more processors (or processor devices) of computer system such as, for example, any general purpose computing system or device such as a personal computer, workstation, cellular phone, smartphone, laptop, tablet, or the like. As such, the computer system may include any suitable hardware and component designed or capable of carrying out a variety of processing and control tasks, including, but not limited to, steps for receiving a input image 102 of the subject, implementing the pre-processing module 104, implementing the acoustic beam profile simulation module 106, implementing the planning module 108, and implementing the real-time navigation module 110. For example, the computer system may include a programmable processor or combination of programmable processors, such as central processing units (CPUs), graphics processing units (GPUs), and the like. In some implementations, the one or more processors of the computer system may be configured to execute instructions stored in a non-transitory computer readable-media. In this regard, the computer system may be any device or system designed to integrate a variety of software, hardware, capabilities, and functionalities. Alternatively, and by way of particular configurations and programming, the computer system may be a special-purpose system or device. For instance, such special purpose system or device may include one or more dedicated processing units or modules that may be configured (e.g., hardwired, or pre-programmed) to carry out steps, in accordance with aspects of the present disclosure.

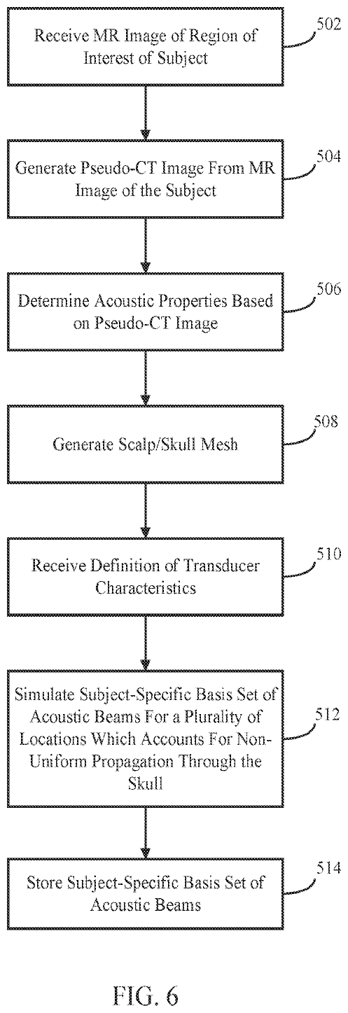

FIG. 6 illustrates an example method for determining acoustic beam profiles in accordance with an embodiment. The process illustrated in FIG. 6 is described below as being carried out by the system 100 for planning and real-time navigation for transcranial focused ultrasound stimulation (tFUS) as illustrated in FIG. 1 and the acoustic beam profile simulation module 106 illustrated in FIG. 2. Although the blocks of the process are illustrated in a particular order, in some embodiments, one or more blocks may be executed in a different order than illustrated in FIG. 6, or may be bypassed.

At block 502, an MR image 102 of a region of interest of a subject may be received. The MR image 102 may be acquired using, for example, an MRI system using various MR imaging (MRI) acquisition techniques. In some embodiments, the input MR image 102 of the subject may be retrieved from data storage (or memory) of an imaging system (e.g., an MRI system or a CT system) or data storage of other computer systems (e.g., storage device 916 of computer system 900 shown in FIG. 10). In some embodiments, the subject's MR may be displayed on a display 122 of the system 100 or a display of other computer systems (e.g., display 818 of the computer system 800 shown in FIG. 9). At block 504,. a pseudo-CT image may be generated from the MR image of the subject, for example, using the pre-processing module 104. For example, the MR image 102 may be converted or transformed into CT units (Hounsfield units (HUs) using a pseudo-CT technique to generate a pseudo-CT image that accounts for the subject's individual skull geometry. At block 506, acoustic properties of, for example, the skull of the subject, may be determined based on the pseudo-CT image. In some embodiments, the pre-processing module 104 may be used to determine or estimate acoustic properties of the subject's skull based on the pseudo-CT image. For example, the acoustic properties of the skull may be estimated from the pseudo-CT image by using a scaling method that scales the Hounsfield units to acoustic properties (or parameters) or by using deep learning (e.g., a neural network).

At block 508, a scalp/skull mesh 132 may be generated, for example, using the acoustic beam profile simulation module 106. In some embodiments, the mesh 132 can be a representation of the scalp of the subject based on information from the MR image of the subject. For example, the scalp surface mesh 132 may be generated using a meshing routine. In some embodiments, the vertices of scalp/skull mesh 132 can represent the test transducer locations to be solved for by the acoustic beam profile simulation module 106 (e.g., using an acoustic beam profile solver 136). In some embodiments, around 1000 vertices can be used. More vertices may result in longer pre-computation times. In some embodiments, a user may select the number of vertices of the mesh 132 and may select a region outside of which vertices of the mesh are removed. For example, a user may manually select a 3D “box” outside of which mesh vertices may be rejected to avoid calculation of acoustic beams at locations that are not accessible to the operator or not relevant. In some embodiments, a smoothing technique (e.g., spatial smoothing may be used when generated the scalp surface mesh 132. At block 510, a definition 134 of the transducer characteristics may be received or a set of basis functions characteristics may be received. In some embodiments, for generating the set of acoustic beam profiles for a plurality of transducer locations. the transducer definition 134 may be provided or selected, for example, by a user or operator, that includes a plurality of transducer characteristics or parameters. In some embodiments, where the set of acoustic beam profiles are decomposed on a basis set of ultrasound excitations, a set of basis function characteristics 135 may be provided or selected, for example, by a user or operator.

At block 512, a subject specific basis set of acoustic beams (e.g., beam profiles) 138 for a plurality of locations may be simulated or generated, for example, using the acoustic beam profile solver 136 of the acoustic beam profile simulation module 106. In some embodiments, the transducer profiles are determined at hundreds of locations around the subject's scalp. In some embodiments, a fast method for computation of the tFUS beam profiles may be used, for example, a hybrid angular spectrum (HAS) method, a finite element difference time domain accelerated on a graphical processing unit (GPU), etc. Advantageously, the basis set of acoustic beams 138 accounts for non-linear acoustic propagation effects of structures, for example, the skull of the subject. In some embodiments, the acoustic beam profile solver 136 computes acoustic beams for a plurality of transducer locations around the subject's scalp based on, for example, the acoustic properties 130, the scalp mesh 132, and the transducer definition 134. . . . In some embodiments, the acoustic beam profile solver 136 computes acoustic beams for a basis set of ultrasound excitations (e.g., point sources, random sources, plane waves, etc.) at plurality (e.g., hundreds) of locations around the scalp based on, for example, the acoustic properties 130, the scalp mesh 132, and the basis function characteristics 135. Other transducer excitation sources can be used to create the basis set of ultrasound excitations, for example, random sources and plane waves which can be used for decomposition of arbitrary acoustic field produced by sources placed outside the head. At block 514, the subject-specific basis set of acoustic beams 138 may be stored in data storage (or memory) 120 of system 100 or data storage of other computer systems (e.g., storage device 916 of computer system 900 shown in FIG. 10).

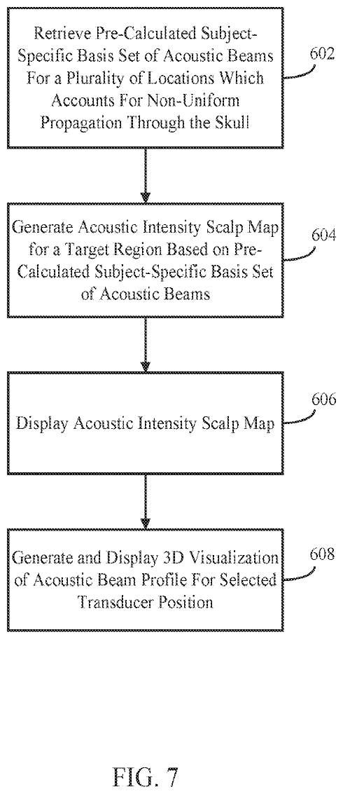

FIG. 7 illustrates method for planning for transcranial focused ultrasound stimulation (tFUS) study for a subject in accordance with an embodiment. The process illustrated in FIG. 7 is described below as being carried out by the system 100 for planning and real-time navigation for transcranial focused ultrasound stimulation (tFUS) as illustrated in FIG. 1. Although the blocks of the process are illustrated in a particular order, in some embodiments, one or more blocks may be executed in a different order than illustrated in FIG. 7, or may be bypassed.

At block 602, the pre-calculated subject-specific basis set of acoustic beams for a plurality of locations may be retrieved by a planning tool 108. In some embodiments, the subject-specific basis set of acoustic beams may be retrieved from data storage (or memory) 120 of system 100 or data storage of other computer systems (e.g., storage device 816 of computer system 800 shown in FIG. 9). At block 604, an acoustic intensity scalp map 112 for a target region may be generated based on the pre-calculated subject-specific basis set of acoustic beams. In some embodiments, the scalp map for a target region can show the acoustic intensity in the brain target of interest for all of the transducer locations (or point source locations) simulated or modeled by the acoustic beam profile simulation module 106. The brain target or region of interest can depend on the specific tFUS study (or application) to be conducted for the subject, for example, some application may target the thalamus, other applications may target the amygdala, etc. In some embodiments, the target region can be selected or defined by a user. In some embodiments, a segmentation method may be used to segment regions that may be used as the target, for example, various cortical and subcortical regions. At block 706, the acoustic intensity scalp map 112 may be displayed. In some embodiments, the acoustic intensity scalp map 112 may be displayed on a display 122 of the system 100 or a display of other computer systems (e.g., display 818 of the computer system 800 shown in FIG. 9).

At block 608, a 3D visualization 114 of an acoustic beam profile for a selected transducer position may be generated and displayed. In some embodiments, the planning modules 108 can allow a user to freely move a virtual transducer around a 3D scalp representation of the subject (e.g., a 3D mesh representation of the subject's scalp generated by the acoustic beam profile simulation module 106) and visualize 114 the previously calculated 3D beam profiles and acoustic focusing performance for specific transducer locations. Accordingly, in some embodiments, a visualization 114 of the acoustic 3D beam profile (i.e., from the subject-specific basis set of acoustic beam profiles) for any selected transducer location may be generated. In some embodiments, the 3D visualization 114 of an acoustic beam profile for a selected transducer position may be displayed on a display 122 of the system 100 or a display of other computer systems (e.g., display 818 of the computer system 800 shown in FIG. 9).

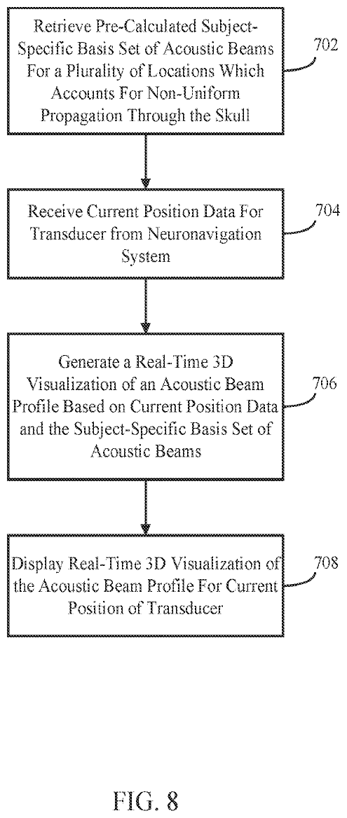

FIG. 8 illustrates a method for real-time navigation for a transcranial focused ultrasound stimulation (tFUS) in accordance with an embodiment. The process illustrated in FIG. 8 is described below as being carried out by the system 100 for planning and real-time navigation for transcranial focused ultrasound stimulation (tFUS) as illustrated in FIG. 1. Although the blocks of the process are illustrated in a particular order, in some embodiments, one or more blocks may be executed in a different order than illustrated in FIG. 8 or may be bypassed.

At block 702, the pre-calculated subject-specific basis set of acoustic beams for a plurality of locations may be retrieved by a navigation system 110. In some embodiments, the subject-specific basis set of acoustic beams may be retrieved from data storage (or memory) 120 of system 100 or data storage of other computer systems (e.g., storage device 816 of computer system 800 shown in FIG. 9). At block 704, current position data (e.g., position and orientation) for a physical transducer of a focused ultrasound system may be received, for example, from a neuronavigation system 118. In some embodiments, the position data may be determined by tracking the movements of the physical transducer around the subject's scalp, for example, by using an optical neuronavigation tracking system. At block 706, a real-time 3D visualization 116 of an acoustic beam profile based on current position data and the subject-specific basis set of acoustic beams. In some embodiments, the real-time display (or visualization) 116 of the tFUS beam as deformed by the skull, may be generated as the operator moves the transducer around the subject's scalp. At block 708, the real-time 3D visualization 116 of an acoustic beam profile for the current position of the transducer may be displayed. In some embodiments, the real-time 3D visualization of an acoustic beam profile for the current position of the transducer may be displayed on a display 122 of the system 100 shown in FIG. 1 or a display of other computer systems (e.g., display 818 of the computer system 800 shown in FIG. 9).

FIG. 9 is a block diagram of an example computer system in accordance with an embodiment. Computer system 800 may be used to implement the systems and methods described herein. In some embodiments, the computer system 800 may be a workstation, a notebook computer, a tablet device, a mobile device, a multimedia device, a network server, a mainframe, one or more controllers, one or more microcontrollers, or any other general-purpose or application-specific computing device. The computer system 800 may operate autonomously or semi-autonomously, or may read executable software instructions from the memory or storage device 816 or a computer-readable medium (e.g., a hard drive, a CD-ROM, flash memory), or may receive instructions via the input device 822 from a user, or any other source logically connected to a computer or device, such as another networked computer or server. Thus, in some embodiments, the computer system 800 can also include any suitable device for reading computer-readable storage media.

Data, such as data acquired with an imaging system (e.g., a CT imaging system, a magnetic resonance imaging (MRI) system, etc.) or a neuronavigation system may be provided to the computer system 800 from a data storage device 816, and these data are received in a processing unit 802. In some embodiment, the processing unit 802 includes one or more processors. For example, the processing unit 802 may include one or more of a digital signal processor (DSP) 804, a microprocessor unit (MPU) 806, and a graphics processing unit (GPU) 808. The processing unit 802 also includes a data acquisition unit 810 that is configured to electronically receive data to be processed. The DSP 804, MPU 806, GPU 808, and data acquisition unit 810 are all coupled to a communication bus 812. The communication bus 812 may be, for example, a group of wires, or a hardware used for switching data between the peripherals or between any components in the processing unit 802.

The processing unit 802 may also include a communication port 814 in electronic communication with other devices, which may include a storage device 816, a display 818, and one or more input devices 820. Examples of an input device 820 include, but are not limited to, a keyboard, a mouse, and a touch screen through which a user can provide an input. The storage device 816 may be configured to store data, which may include data such as, for example, MRI images, pseudo-CT images, CT images, scalp/skull mesh, transducer characteristics, acoustic beam profiles, acoustic intensity scalp maps, 3D beam profile visualizations, etc., whether these data are provided to, or processed by, the processing unit 802. The display 818 may be used to display images and other information, such as magnetic resonance images, patient health data, and so on.

The processing unit 802 can also be in electronic communication with a network 822 to transmit and receive data and other information. The communication port 814 can also be coupled to the processing unit 802 through a switched central resource, for example the communication bus 812. The processing unit can also include temporary storage 824 and a display controller 826. The temporary storage 824 is configured to store temporary information. For example, the temporary storage 824 can be a random access memory.

As mentioned above, the disclosed systems and methods may be implemented for the planning and real-time navigation for tFUS which can be performed using a focused ultrasound (“FUS”) system. FIG. 10 is block diagram of an example focused ultrasound system in accordance with an embodiment. Ultrasound system 900 may be configured to perform and deliver focused ultrasound (“FUS”). The ultrasound system 900 generally includes a transducer 902 that is capable of delivering ultrasound to a subject 904 and receiving responsive signal therefrom. Transducer 902 may be a single-element transducer or an arrayed transducer. The transducer 902 may be configured to be a shape and size appropriate for delivering focused ultrasound energy to a desired region of interest 906 (e.g., a particular tissue or organ) in the subject 904. For example, for brain applications the transducer 902 may be an approximately hemispherical array of transducer elements or a single-element transducer configured to surround a portion of the subjects head (or scalp).

The ultrasound system 900 also includes a controller (or processor) 908 that is in communication with a transmitter 910 and a receiver 912. The transmitter 910 receives driving signals from the controller 908 and, in turn, directs the transducer elements of the transducer 902 to generate ultrasound energy (or acoustic pressure waves). In an embodiment, the transmitter may include a power amplifier (not shown) and impedance matching circuit (not shown) to amplify signals before transmitting them to the transducer 902. The acoustic pressure waves generated by the transducer 902 are delivered to the region of interest 906 (or target region or target site) via acoustic coupling between the transducer 902 and the subject using various media such as, for example, water or hydrogel. The acoustic intensity (i.e., the acoustic power per given area (W/cm²) is expressed in spatial-peal pulse-average intensity (Iₛₚₚₐ) while Iₛₚₜₐ represents its time-average value per each stimulus. In an embodiment, the focused ultrasound delivered by transducer 902 may be given in a batch of pulsed sinusoidal or square pressure waves at a fundamental frequency (FF). The individual pulses each have a specific toe-burst duration (TBD) and are administered in a repeated fashion with a pulse repetition frequency (PRF). The duty cycle of sonication (in %) may be determined by multiplying the TBD by the PRF. The duty cycle indicates the fraction of active sonication time per each sonication. The overall duration of the pulsed sonication is termed sonication duration.

The receiver 912 receives acoustic signals during and/or after sonication and relays these signals to the controller 908 for processing. The controller 908 may also be configured to adjust the driving signals in response to the acoustic emissions recorded by the receiver 912. For example, the phase and/or amplitude of the driving signals may be adjusted so that ultrasound energy is more efficiently transmitted through, for example, the skin and/or the skull of the subject 904 and into the target region of interest (or target region or target site) 906. Furthermore, the acoustic signals may also be analyzed to determine whether and how the extent of the focal region should be adjusted. In an embodiment, an image guided system 920 may be used to navigate the acoustic focus to the region of interest 906. The image guided system 920 may be, for example, MR, fMRI or computer tomography (CT). The image guided system 920 may be co-registered with the physical space using known methods. In another embodiment, numerical acoustic simulation may be used to estimate the location and intensity of the acoustic focus.

The ultrasound system 900 may also include a user input 914, data storage 916 and a display 918 which are coupled to the controller 908. User input 914 may include one or more input devices (such as a keyboard and a mouse, or the like) configured for operation of the controller 908, including the ability for selecting, entering or otherwise specific parameters consistent with performing tasks, processing data, or operating the FUS ultrasound system 900. Data storage 916 may contain software and data and may be configured for storage and retrieval of processed information, instructions, and data to be processed. Display 918 may be used to display, for example, data and images.

Computer-executable instructions for subject-specific planning and real-time navigation for transcranial focused ultrasound stimulation (tFUS) using pre-calculated set of acoustic beam profiles that account for non-uniform propagation through the skull according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. A system for planning and real-time navigation for transcranial focused ultrasound stimulation (tFUS) comprises:
an input for receiving an image of a head of a subject;
an acoustic beam profile simulation module coupled to the input and configured to generate a subject-specific set of acoustic beam profiles based on the image of the head of the subject, wherein the subject-specific set of acoustic beam profiles is configured to account for acoustic propagation effects through the subject's skull;
a planning module coupled to the acoustic beam profile simulation module and configured to generate an acoustic intensity scalp map for a target region based on the subject-specific set of acoustic beam profiles and to generate a three-dimensional (3D) visualization of a selected beam profile from the subject-specific set of acoustic beam profiles; and
a real-time navigation module coupled to the acoustic beam profile simulation module and configured to generate a real-time 3D visualization of an acoustic beam for tFUS for a current position of a transducer around the head of the subject based on current position data and the subject-specific set of acoustic beam profiles.

2. The system according to claim 1, further comprising a display configured to display one or more of the image of the head of the subject, the scalp map, the 3D visualization of a selected beam profile from the subject-specific set of acoustic beam profiles, and the real-time 3D visualization of an acoustic beam for tFUS for a current position of a transducer around the head of the subject.

3. The system according to claim 1, wherein the image of the head of the subject is a magnetic resonance (MR) image.

4. The system according to claim 2, further comprising a pre-processing module couped to the input and the acoustic beam profile simulation module, the pre-processing module configured to convert the MR image of the subject to a pseudo-CT image and to determine a set of acoustic properties of the skull of the subject based on the pseudo-CT image.

5. The system according to claim 4, wherein the acoustic beam profile simulation module is further configured to generate a scalp mesh having a plurality of vertices representing transducer locations around the head of the subject.

6. The system according to claim 1, wherein the acoustic beam profile simulation module is configured to generate the subject-specific set of acoustic beam profiles for a plurality of transducer locations around the head of the subject.

7. The system according to claim 1, wherein the acoustic beam profile simulation module is configured to generate a basis set of ultrasound excitations and decompose the subject-specific set of acoustic beam profiles on the basis set of ultrasound excitations. 8 The system according to claim 1, wherein the current position data for the transducer is received from a neuronavigation system configured to track the position of the transducer.

9. The system according to claim 3, wherein the real-time navigation module is further configured to register the head of the subject to the image of the head of the subject.

10. A method for planning a transcranial focused ultrasound stimulation (tFUS) study for a subject, the method comprising:
retrieving a pre-calculated subject-specific set of acoustic beam profiles for a plurality of transducer locations, wherein the subject-specific set of acoustic beam profiles is configured to account for acoustic propagation effects through the subject's skull;
generating an acoustic intensity scalp map for a target region based on the subject-specific set of acoustic beam profiles;
generating a three-dimensional (3D) visualization for a selected beam profile from the subject-specific set of acoustic beam profiles; and
displaying the acoustic intensity scalp map and the 3D visualization of the selected beam profile on a display.

11. The method according to claim 10, wherein the subject-specific set of acoustic beam profile is generated based on a magnetic resonance image of a head of the subject.

12. The method according to claim 10, the target region is a region of a brain of the subject.

13. A method for real-time navigation for a transcranial focused ultrasound stimulation (tFUS) study of a subject, the method comprising:
retrieving a pre-calculated subject-specific set of acoustic beam profiles for a plurality of transducer locations, wherein the subject-specific set of acoustic beam profiles is configured to account for acoustic propagation effects through the subject's skull;
receiving current position data for a transducer indicating a current position of the transducer around the head of the subject;
generating a real-time 3D visualization of an acoustic beam for tFUS for the current position of a transducer around the head of the subject based on the current position data and the subject-specific set of acoustic beam profiles; and
displaying the real-time 3D visualization of an acoustic beam for tFUS for the current position of a transducer around the head of the subject.

14. The method according to claim 13, wherein the subject-specific set of acoustic beam profile is generated based on a magnetic resonance image of a head of the subject.

15. The method according to claim 13, wherein the current position data for the transducer is received from a neuronavigation system configured to track the position of the transducer.

16. The method according to claim 14, further comprising registering the head of the subject to the MR image of the head of the subject.
